(19) United States
(12) Patent Application Publication
(10) Pub. No.: US 2024/0047055 A1
(43) Pub. Date: Feb. 8, 2024

(54) METHOD AND SYSTEM FOR MEDICAL CODING AND BILLING

(71) Applicant: The Board of Regents for the Oklahoma Agricultural and Mechanical Colleges, Stillwater, OK (US)

(72) Inventors: Suhao Chen, Stillwater, OK (US); Zhuqi Miao, Stillwater, OK (US); Thanh Thieu, Stillwater, OK (US); Andrew Gin, Stillwater, OK (US); William Paiva, TULSA, OK (US)

(21) Appl. No.: 18/481,822

(22) Filed: Oct. 5, 2023

Related U.S. Application Data
(63) Continuation of application No. PCT/US22/24857, filed on Apr. 14, 2022.
(60) Provisional application No. 63/174,907, filed on Apr. 14, 2021.

(51) Int. Cl. G16H 40/20; G16H 10/60

(52) U.S. Cl. CPC G16H 40/20; G16H 10/60

(57) ABSTRACT

A system and method configured to segment computer readable clinical notes of a healthcare provider into a plurality of sections having textual content. For each section, the system is configured to parse the textual content with natural language processing using at least one pre-defined dictionary. A set of predefined rules are applied to the parsed textual content to generate at least one candidate. The at least one candidate provides at least one current procedural terminology (CPT) medical code based on the parsed textual content.

### CROSS REFERENCE TO RELATED APPLICATIONS/INCORPORATION BY REFERENCE STATEMENT

The present patent application is a continuation and claims priority to the patent application identified by International Application No. PCT/US22/24857, filed Apr. 14, 2022; which claims priority to the provisional patent application identified by U.S. Ser. No. 63/174,907, filed on Apr. 14, 2021. The entire content of both patent application is hereby incorporated herein by reference.

### STATEMENT REGARDING FEDERALLY SPONSORED RESEARCH OR DEVELOPMENT

Not Applicable.

### BACKGROUND

Medical billing is critical to revenue cycle management of healthcare providers with a market size estimated to be in the billions of dollars. With correct and efficient medical billing, healthcare providers are able to be reimbursed for professional medical services. However, current methods used in medical billing are prone to errors and time-consuming.

Generally, medical billing involves the use of medical coding that relies heavily on Current Procedural Terminology (CPT) codes. The American Medical Association assigns a unique 5-digit code to each unique medical treatment or procedure a doctor provides. In practice, initially, a doctor sees a patient. Based on the interaction between the doctor and patient, the healthcare provider initiates the coding process. If the doctor uses a paper encounter form, the doctor will manually note which CPT code applied to the visit between the doctor and patient. If the doctor uses an electronic health record (EHR), the clinical notes may be provided to a processor for identification of the CPT code.

CPT codes are distinct from ICD-10 codes. ICD-10 codes identify medical diagnosis rather than the treatment performed. CPT codes have a direct impact on how much a patient pays for medical services. The CPT codes assist insurance companies, for example, to determine whether coverage exists for that particular patient. The medical claim, having the one or more codes, is submitted to a payer or clearinghouse that can process the medical claim usually by evaluation by a medical claim examiner and/or a medical claim adjuster.

For decades, medical billing using medical coding was provided solely via paper. With the rise of the computer age, such practice has been moved and assisted by medical practice management software. Such software, however, is still prone to errors. Most existing computer-assisted medical billing technologies currently focus on basic processes and provide a broad human-computer interaction framework for application of computer-assisted coding in the medical billing domain. For example, such systems and methods can be found in U.S. Pat. No. 10,319,004, entitled, “User and engine code handling in medical coding system,” U.S. Pat. No. 10,373,711, entitled, “Medical coding system with CDI clarification request notification,” and U.S. Patent Publication No. 2006/0173778, entitled, “Enterprise billing system for medical billing.” Some prior art systems provide specific models without providing implementation direction. For example, such systems and methods can be found in U.S. Patent Publication No. 2007/0050187, entitled, “Medical billing system and method,” and U.S. Pat. No. 6,915,254, entitled, “Automatically assigning medical codes using natural language processing.” Other systems propose to use computer visualization tools to aid human coders. For example, such systems and methods can be found in U.S. Pat. No. 10,366,424, entitled, “Medical coding system with integrated codebook interface,” relating to use of a coding user interface, and U.S. Pat. No. 8,666,772, entitled, “Process, system, method creating medical billing code letters, electronic superbill and communication,” relating to use of machine-readable code letters.

Correct medical billing requires an appropriate depiction of patient conditions, treatments and tests. Currently, clinical notes are provided via doctors in unstructured formats. Reading such notes provided by doctors is tedious and challenging even for experienced human medical coders. Hence, it is important to develop a computer-assisted coding system that can relieve human coders from such burdens and help healthcare providers reduce billing errors.

### BRIEF DESCRIPTION OF THE DRAWINGS

FIG. 1 is a block diagram of an exemplary embodiment of a medical coding and billing system in accordance with the present disclosure.

FIG. 2 is a block diagram of an exemplary embodiment of a medical coding system for use in the medical coding and billing system illustrated in FIG. 1.

FIG. 3A1 depicts a first portion of an exemplary segmented clinical note in accordance with the present disclosure.

FIG. 3A2 depicts a second portion of an exemplary segmented clinical note in accordance with the present disclosure.

FIG. 3A3 depicts a third portion of an exemplary segmented clinical note in accordance with the present disclosure.

FIG. 3B illustrates exemplary output obtained from the exemplary segmented clinical note in FIGS. 3A1-3 in accordance with the present disclosure.

FIG. 4 is a block diagram of another exemplary embodiment of a medical coding system for use in the medical coding and billing system illustrated in FIG. 1.

FIG. 5 is a flow chart of an exemplary method for identifying at least one candidate using at least one predefined dictionary in accordance with the present disclosure.

FIG. 6 is a flow chart of an exemplary method for identifying at least one candidate in a plurality of sections in accordance with the present disclosure.

FIG. 7 is a flow chart of an exemplary method for identifying at least one candidate using a plurality of predefined dictionaries in accordance with the present disclosure.

FIG. 8 is a flow chart of an exemplary method for applying a deep learning model to further aid in identification of one or more entities and development of one or more candidate lists to provide one or more medical codes in accordance with the present disclosure.

### DETAILED DESCRIPTION

Before explaining at least one embodiment of the inventive concept(s) in detail by way of exemplary language and results, it is to be understood that the inventive concept(s) is not limited in its application to the details of construction and the arrangement of the components set forth in the following description. The inventive concept(s) is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

Unless otherwise defined herein, scientific and technical terms used in connection with the presently disclosed inventive concept(s) shall have the meanings that are commonly understood by those of ordinary skill in the art. Further, unless otherwise required by context, singular terms shall include pluralities and plural terms shall include the singular. The foregoing techniques and procedures are generally performed according to conventional methods well known in the art and as described in various general and more specific references that are cited and discussed throughout the present specification.

All patents, published patent applications, and non-patent publications mentioned in the specification are indicative of the level of skill of those skilled in the art to which this presently disclosed inventive concept(s) pertains. All patents, published patent applications, and non-patent publications referenced in any portion of this application are herein expressly incorporated by reference in their entirety to the same extent as if each individual patent or publication was specifically and individually indicated to be incorporated by reference.

All of the compositions, assemblies, systems, kits, and/or methods disclosed herein can be made and executed without undue experimentation in light of the present disclosure. While the compositions, assemblies, systems, kits, and methods of the inventive concept(s) have been described in terms of particular embodiments, it will be apparent to those of skill in the art that variations may be applied to the compositions and/or methods and in the steps or in the sequence of steps of the methods described herein without departing from the concept, spirit, and scope of the inventive concept(s). All such similar substitutions and modifications apparent to those skilled in the art are deemed to be within the spirit, scope, and concept of the inventive concept(s) as defined by the appended claims.

As utilized in accordance with the present disclosure, the following terms, unless otherwise indicated, shall be understood to have the following meanings:

The use of the term “a” or “an” when used in conjunction with the term “comprising” in the claims and/or the specification may mean “one,” but it is also consistent with the meaning of “one or more,” “at least one,” and “one or more than one.” As such, the terms “a,” “an,” and “the” include plural referents unless the context clearly indicates otherwise. Thus, for example, reference to “a compound” may refer to one or more compounds, two or more compounds, three or more compounds, four or more compounds, or greater numbers of compounds. The term “plurality” refers to “two or more.”

The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z. The use of ordinal number terminology (i.e., “first,” “second,” “third,” “fourth,” etc.) is solely for the purpose of differentiating between two or more items and is not meant to imply any sequence or order or importance to one item over another or any order of addition, for example.

The use of the term “or” in the claims is used to mean an inclusive “and/or” unless explicitly indicated to refer to alternatives only or unless the alternatives are mutually exclusive. For example, a condition “A or B” is satisfied by any of the following: A is true (or present) and B is false (or not present), A is false (or not present) and B is true (or present), and both A and B are true (or present).

As used herein, any reference to “one embodiment,” “an embodiment,” “some embodiments,” “one example,” “for example,” or “an example” means that a particular element, feature, structure, or characteristic described in connection with the embodiment is included in at least one embodiment. The appearance of the phrase “in some embodiments” or “one example” in various places in the specification is not necessarily all referring to the same embodiment, for example. Further, all references to one or more embodiments or examples are to be construed as non-limiting to the claims.

Throughout this application, the term “about” is used to indicate that a value includes the inherent variation of error for a composition/apparatus/device, the method being employed to determine the value, or the variation that exists among the study subjects. For example, but not by way of limitation, when the term “about” is utilized, the designated value may vary by plus or minus twenty percent, or fifteen percent, or twelve percent, or eleven percent, or ten percent, or nine percent, or eight percent, or seven percent, or six percent, or five percent, or four percent, or three percent, or two percent, or one percent from the specified value, as such variations are appropriate to perform the disclosed methods and as understood by persons having ordinary skill in the art.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, the term “substantially” means that the subsequently described event or circumstance completely occurs or that the subsequently described event or circumstance occurs to a great extent or degree. For example, when associated with a particular event or circumstance, the term “substantially” means that the subsequently described event or circumstance occurs at least 80% of the time, or at least 85% of the time, or at least 90% of the time, or at least 95% of the time. For example, the term “substantially adjacent” may mean that two items are 100% adjacent to one another, or that the two items are within close proximity to one another but not 100% adjacent to one another, or that a portion of one of the two items is not 100% adjacent to the other item but is within close proximity to the other item.

As used herein, the phrases “associated with” and “coupled to” include both direct association/binding of two moieties to one another as well as indirect association/binding of two moieties to one another. Non-limiting examples of associations/couplings include covalent binding of one moiety to another moiety either by a direct bond or through a spacer group, non-covalent binding of one moiety to another moiety either directly or by means of specific binding pair members bound to the moieties, incorporation of one moiety into another moiety such as by dissolving one moiety in another moiety or by synthesis, and coating one moiety on another moiety, for example.

Circuitry, as used herein, may be analog and/or digital components, or one or more suitably programmed processors (e.g., microprocessors) and associated hardware and software, or hardwired logic. Also, “components” may perform one or more functions. The term “component,” may include hardware, such as a processor (e.g., microprocessor), an application specific integrated circuit (ASIC), field programmable gate array (FPGA), a combination of hardware and software, and/or the like. The term “processor” as used herein means a single processor or multiple processors working independently or together to collectively perform a task.

Software may include one or more computer readable instructions that when executed by one or more components cause the component to perform a specified function. It should be understood that the algorithms described herein may be stored on one or more non-transitory memory. Exemplary non-transitory memory may include random access memory, read only memory, flash memory, and/or the like. Such non-transitory memory may be electrically based, optically based, and/or the like.

The term “healthcare provider” as used herein includes a person or group of persons capable of providing health services including, but not limited to, a doctor of medicine or osteopathy, podiatrist, dentist, chiropractor, clinical psychologist, optometrist, nurse practitioner, nurse-midwife, nurse, a clinical social worker, veterinarian and the like. Further, “healthcare provider” may include any provider whom an insurance provider will accept medical codes to substantiate a claim for benefits.

The term “patient” as used herein includes human and veterinary subjects.

The term “Evaluation and Management medical codes” or “E/M medical codes” as used herein refers to the numeric coding system current procedural terminology (CPT) codes maintained by the American Medical Association (AMA). The CPT coding system is a uniform coding system consisting of descriptive terms and identifying codes to identify medical services and procedures furnished by healthcare provides.

Certain exemplary embodiments of the invention will not be described with reference to the drawings. In general, such embodiments relate to systems and methods for computer-assisted medical billing.

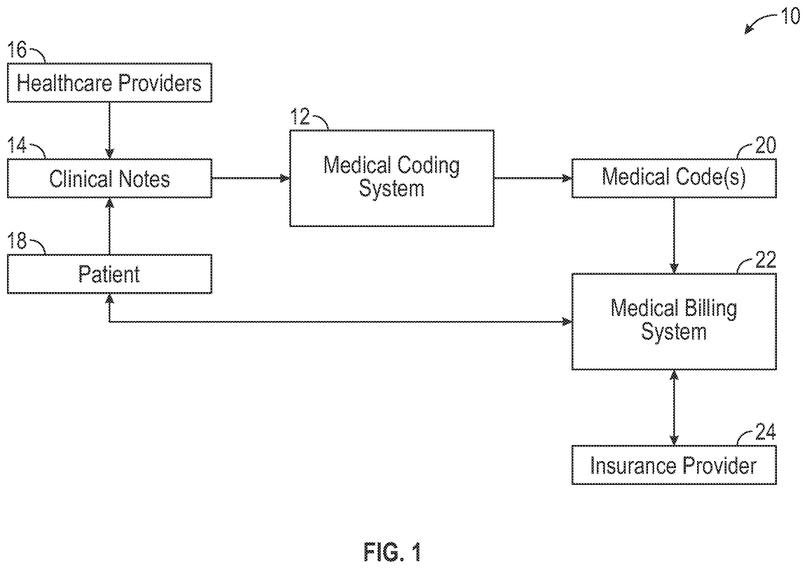

Turning now to the drawing and in particular to FIG. 1, certain non-limiting embodiments thereof include a medical evaluation, coding and billing system 10 having a medical coding system 12 configured to obtain one or more clinical notes 14 from one or more healthcare providers 16 providing one or more services and/or evaluation(s) of a patient 18. The medical coding system 12 provides one or more Evaluation and Management (E/M) medical codes 20 determined from the one or more clinical notes 14 via natural language processing, expert knowledge of E/M coding protocols, rule-based algorithms, deep learning models, and/or an ensemble scheme as described in further detail herein. Generally, the one or more medical codes 20 may be provided to one or more medical billing systems 22 configured to coordinate between healthcare provider 16, patients 18, and/or insurance providers 24 to obtain payment for services rendered by the healthcare provider 16 to the patient 18.

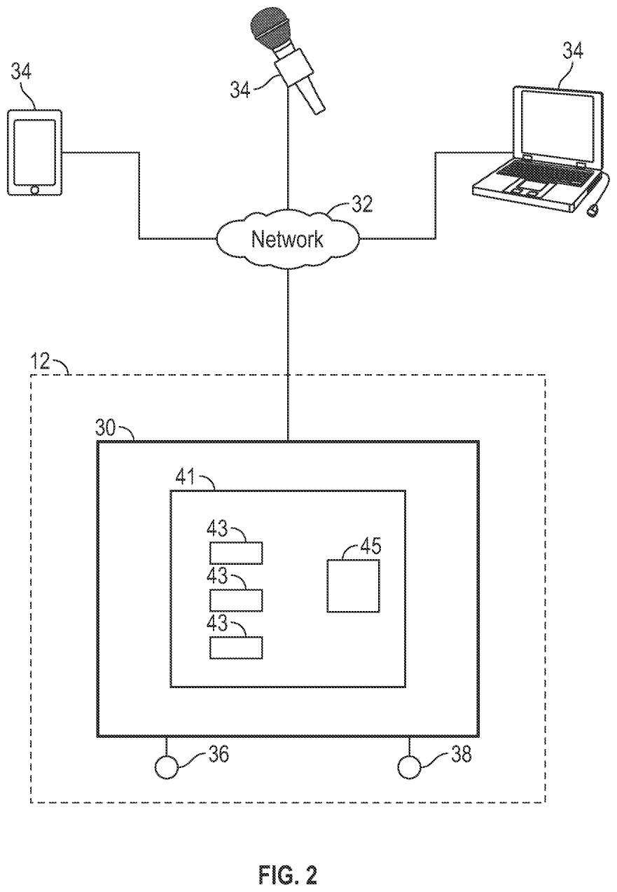

Referring to FIGS. 1 and 2, the medical coding system 12 may be a system or systems that are able to embody and/or execute the logic of the processes described herein. Logic embodied in the form of software instructions and/or firmware may be executed on any appropriate hardware. For example, logic embodied in the form of software instructions or firmware may be executed on a system or systems, or on a personal computer system, or on a distributed processing computer system, and/or the like. In some embodiments, logic may be implemented in a stand-alone environment operating on a single computer system and/or logic may be implemented in a networked environment, such as a distributed system using multiple computers and/or processors networked together.

In some embodiments, the medical coding system 12 may include one or more processors 30. The one or more processors 30 may work to execute processor executable code. The one or more processors 30 may be implemented as a single or plurality of processors working together, or independently, to execute the logic as described herein. Exemplary embodiments of the one or more processors 30 may include, but are not limited to, a digital signal processor (DSP), a central processing unit (CPU), a field programmable gate array (FPGA), a microprocessor, a multi-core processor, and/or combinations thereof, for example. In some embodiments, the one or more processors 30 may be incorporated into a smart device. The one or more processors 30 may be capable of communicating via a network 32 or a separate network (e.g., analog, digital, optical, and/or the like). It is to be understood, that in certain embodiments, using more than one processor, the processors 30 may be located remotely from one another, in the same location, or comprising a unitary multi-core processor. In some embodiments, the one or more processors 30 may be partially or completely network-based or cloud-based, and may or may not be located in a single physical location. The one or more processors 30 may be capable of reading and/or executing processor executable code and/or capable of creating, manipulating, retrieving, altering, and/or storing data structure into one or more memories.

In some embodiments, the one or more processors 30 may transmit and/or receive data via the network 32 to and/or from one or more external systems 34 (e.g., one or more external computer systems, one or more machine learning applications, artificial intelligence, cloud based system, microphones). For example, the one or more processors 30 may allow users (e.g., healthcare providers, physicians, medical personnel, medical billing system) of the external systems 34 access via the network 32 to provide and/or receive data, such as the clinical note(s) 14 and/or medical code(s) 20. Access methods include, but are not limited to, cloud access and direct download to the one or more processors 30 via the network 32. In some embodiments, the one or more processors 30 may be provided on a cloud cluster (i.e., a group of nodes hosted on virtual machines and connected within a virtual private cloud). Additionally, processors 30 may provide data to a user by methods that include, but are not limited to, messages sent through the one or more processors 30 and/or external systems 34, SMS, email, and telephone, to provide data such as positive or negative detection data, for example. It is to be understood that in some exemplary embodiments, the one or more processors 30 and the one or more external systems 34 may be implemented as a single device.

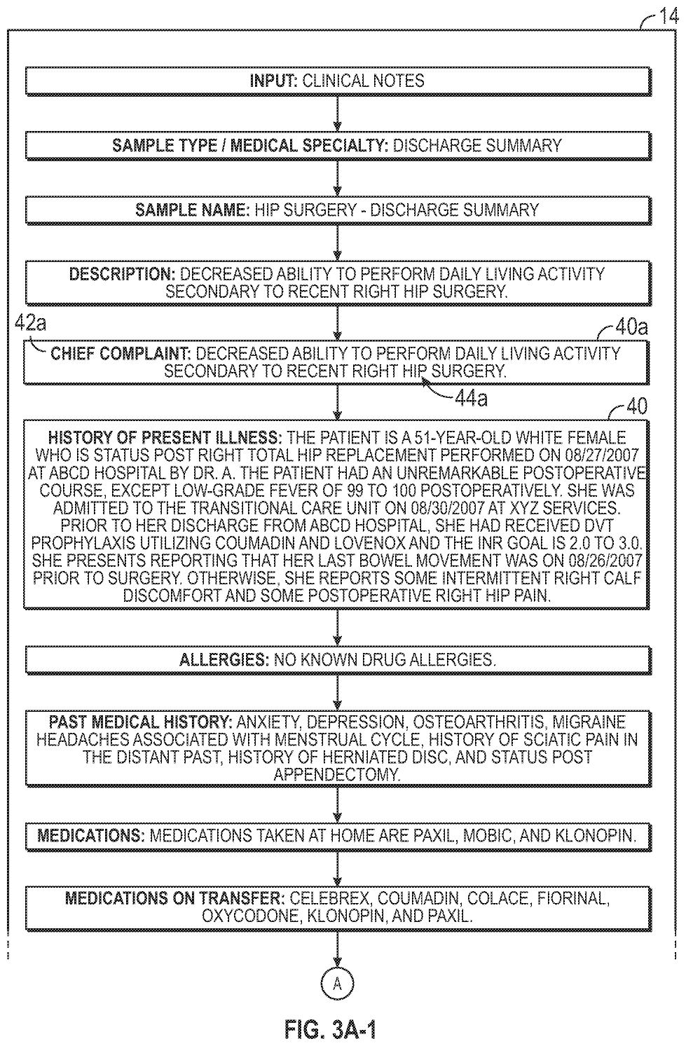

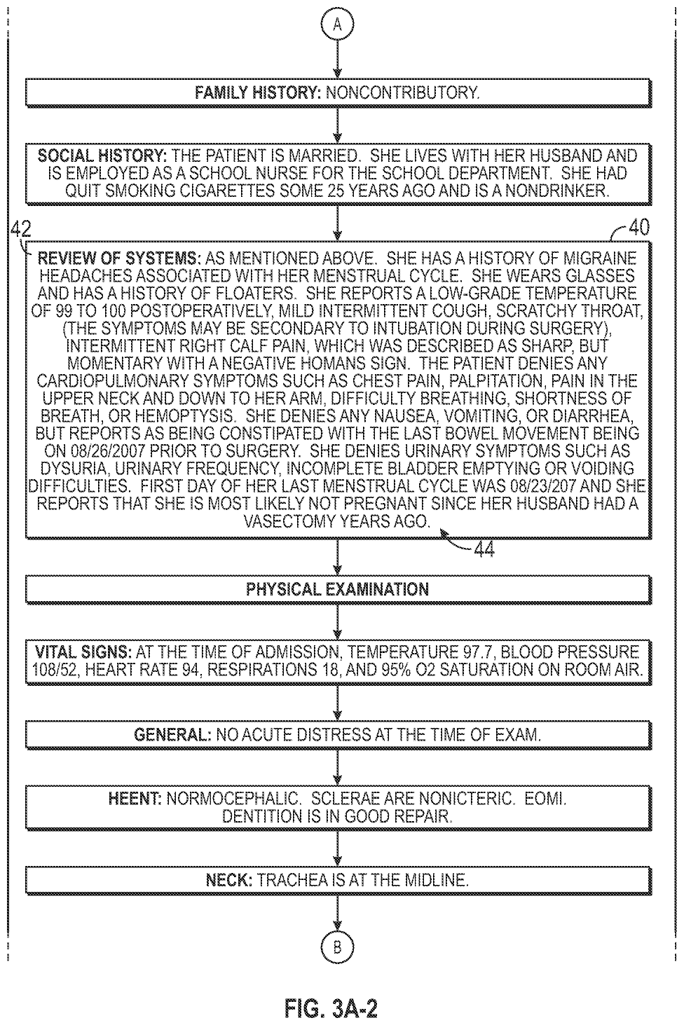

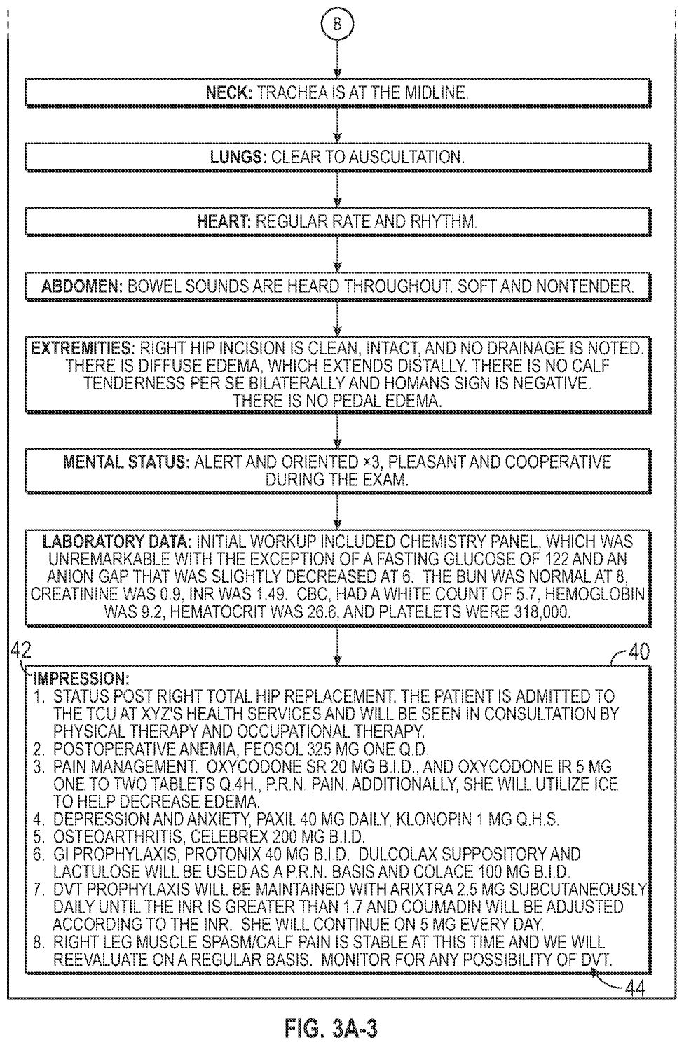

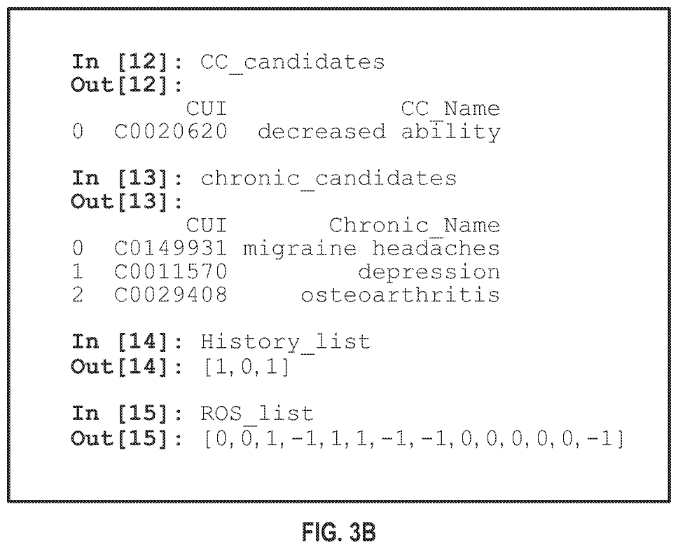

The one or more external systems 34 may be configured to provide information and/or data in a form perceivable to the processors 30. For example, the one or more external systems 34 may include, but are not limited to, implementations as a laptop computer, a computer monitor, a screen, a touchscreen, a microphone, a website, a smart phone, a PDA, a cell phone, an optical head-mounted display, combinations thereof, and/or the like. The externals systems 34 may provide data (e.g., clinical note 14, medical code 20) in computer readable form, such as a text file, a word document, and/or the like. FIG. 3 illustrates an exemplary clinical note 14a in accordance with the present disclosure.

The one or more external systems 34 may communicate with the one or more processors 30 via the network 32. As used herein, the terms “network-based”, “cloud-based”, and any variations thereof, may include the provision of configurable computational resources on demand via interfacing with a computer and/or computer network, with software and/or data at least partially located on a computer and/or computer network, by pooling processing power of two or more networked processors.

In some embodiments, the network 32 may be the Internet and/or other network. For example, if the network 32 is the Internet, a primary user interface of the medical coding software may be delivered through a series of web pages. It should be noted that the primary user interface of the medical billing software may be via any type of interface, such as, for example, a Windows-based application.

The network 32 may be almost any type of network. For example, the network 32 may interface via optical and/or electronic interfaces, and/or may use a plurality of network topographies and/or protocols including, but not limited to, Ethernet, TCP/IP, circuit switched paths, combinations thereof, and the like. For example, in some embodiments, the network 32 may be implemented as the World Wide Web (or Internet), a local area network (LAN), a wide area network (WAN), a metropolitan network, a wireless network, a cellular network, a Global System of Mobile Communications (GSM) network, a code division multiple access (CDMA) network, a 4G network, a 5G network, a satellite network, a radio network, an optical network, an Ethernet network, combinations thereof, and/or the like. Additionally, the network 32 may use a variety of network protocols to permit bi-directional interface and/or communication of data and/or information. It is conceivable that in the near future, embodiments of the present disclosure may use more advanced networking topologies.

In some embodiments, the one or more processors 30 may include one or more input devices 36 and one or more output devices 38. The one or more input devices 36 may be capable of receiving information from a user, processors, and/or environment, and transmit such information to the processor 30 and/or the network 32. The one or more input devices 36 may include, but are not limited to, implementation as a keyboard, touchscreen, mouse, trackball, microphone, fingerprint reader, infrared port, slide-out keyboard, flip-out keyboard, cell phone, PDA, video game controller, remote control, network interface, speech recognition, gesture recognition, combinations thereof, and/or the like. Inputs may be one or more clinical notes 14, for example.

The one or more output devices 38 may be capable of outputting information in a form perceivable by a user, the external system 34, and/or processor(s). For example, the one or more output devices 38 may include, but are not limited to, implementations as a computer monitor, a screen, a touchscreen, a speaker, a website, a television set, a smart phone, a PDA, a cell phone, a fax machine, a printer, a laptop computer, an optical head-mounted display (OHMD), combinations thereof, and/or the like. It is to be understood that in some exemplary embodiments, the one or more input devices 36 and the one or more output devices 38 may be implemented as a single device, such as, for example, a touchscreen or a tablet. Outputs may be one or more Evaluation/Management (E/M) medical codes 20 determined from the inputs via natural language processing, expert knowledge of E/M coding protocols, rule-based algorithms, deep learning models, and/or an ensemble scheme as described in further detail herein.

The one or more processors 30 may be capable of reading and/or executing processor executable code and/or capable of creating, manipulating, retrieving, altering and/or storing data structures into one or more memories 41. The one or more processors 30 may include one or more non-transient memory comprising processor executable code and/or software application. In some embodiments, the one or more memories 41 may be located in the same physical location as the processor 30. Alternatively, one or more memories 41 may be located in a different physical location as the processor 30 and communicate with the processor 30 via a network, such as the network 32. Additionally, one or more memories 41 may be implemented as a “cloud memory” (i.e., one or more memories may be partially or completely based on or accessed using a network, such as network 32).

The one or more memories 41 may store processor executable code and/or information comprising one or more databases 43 and program logic 45 (i.e., computer executable logic). In some embodiments, the processor executable code may be stored as a data structure, such as a database and/or data table, for example. In some embodiments, one or more database 43 may store one or more predefined dictionaries via the methods described herein. In use, the processor 30 may execute the program logic 45 controlling the reading, manipulation and/or storing of data as detailed in the processes described herein.

Referring to FIGS. 1 and 3A1-3, generally, the healthcare provider 16 provides one or more clinical notes 14 based on evaluation and/or treatment of the patient 18. FIG. 3A1-3 illustrates an exemplary clinical note 14 in accordance with the present disclosure. The clinical note 14a is a transcribed clinical note 14 provided by the healthcare provider 16. Transcription of the clinical note 14a may be via speech to recognition software, conversion of handwritten or typed reports, or the like and include procedures and/or notes to provide one or more files in electronic format representing the evaluation and/or treatment of the patient. In some embodiments, healthcare providers 16 may directly provide electronic reports, procedures and/or notes to the clinical note 14 (e.g., via input device 36).

Referring to FIGS. 1, 3A1-3, and 4, the medical coding system 12 provides a computer-assisted method to provide medical codes 20 directly from clinical notes 14. Generally, the medical coding system 12 includes a pre-classification stage 50 for data provided in the clinical notes 14. The pre-classification stage 50 provides for segmentation of sections 40 including classification of clinical notes 14 into different sections 40 using clinical natural language processing (NLP) tools. Subsequent to pre-classification, the medical coding system 12 extracts essential data (i.e., candidates) within each section 40 using predefined dictionaries and rules as described in further detail herein. In some embodiments, the predefined dictionaries and rules may use a hierarchical process to extract candidates from different sections 40. The extracted data (i.e., candidate(s)) within each section 40 is provided to an evaluation/management (E/M) coding engine for generation of candidate medical codes 20.

Initially, the medical coding system 12 pre-classifies data within the clinical note 14 by section segmentation 52 and entity recognition 54. For example, as illustrated in FIG. 3, the clinical note 14a may be segmented into one or more sections 40. Each section 40 may be defined by a section header 42 and section data 44. For example, in FIG. 3, a first section 40a includes the section header 42a “CHIEF COMPLAINT” with section data 44a stating, “[d]ecreased ability to perform daily living activity secondary to recent right hip surgery.” Each section 40 may include different content within the section data 44 with focus of content based on the section header 42. Each clinical note 14 may include multiple sections 40. Section data 44 may include one or more words, phrases, and/or sentences with focus of content based on the section header 42.

Section segmentation 52 is configured to be a virtual division of the clinical note 14 into multiple, distinct parts (i.e., sections 40). In some embodiments, section segmentation 52 of the clinical note 14 may be provided by parsing textual content within the clinical note 14 with one or more natural language processing (NLP) techniques, such as Clinical Language Annotation, Modeling and Processing (CLAMP), clinical Text Analysis and Knowledge Extraction System (cTAKES), Section Tagging (SecTag), and/or the like. NLP techniques such as those described in U.S. Pat. No. 10,176,890, entitled, “Segmenting and interpreting a document, and relocation document fragments to corresponding sections,” may also be implemented. In some embodiments, for example, section segmentation 52 may identify within the clinical note 14 multiple, discrete sections, such as sections 40 having section headers 42 with section data 44. As such, segmentation may use existing section headers 42 (e.g., chief complaint, history of present illness, medications, vital signs, mental status, and the like) to form the multiple, distinct parts.

The pre-classification stage 50 also includes entity recognition 54. In some embodiments, one or more entities within the sections 40 of the clinical note 14 may be identified, negated or related during the pre-classification stage 50. An entity, as used herein, is any word or series of words that refers to a term in a predefined category (e.g., disease entity, medical test entity, chronic condition entities, present problem entity, and the like). For example, the term “diabetes” is a disease entity. The term “A1C blood test” is a medical test entity. Identification of entities, negation of entities, and relation of entities within segmented sections 40 may be performed by parsing textual content with each section 40 using NLP tools such as cTakes, CLAMP, MetaMap, and/or the like. The NLP tools within the medical coding system 12 may identify one or more entities within the section data 44 of the clinical notes 14.

Subsequent to the pre-classification stage 50, the medical coding system 12 may parse textual content within sections 40 of the clinical note 14 using a rule-based natural language processing (NLP) stage 60 using one or more predefined dictionaries, a deep learning based natural language processing stage 64, and an ensemble natural language processing (NLP) model 62 to further classify the one or more entities within the textual content of the clinical note 14. Generally, the medical coding system 12 extracts essential data (i.e., candidates) within each section 40 using predefined dictionaries and rules. Such extracted data (i.e., candidate(s)) is provided to an Evaluation/Management (E/M) coding system configured to generate medical codes 20.

In some embodiments, the rule based NLP stage 60 may further parse the textual content and classify the one or more entities into one or more candidates using one or more predefined dictionaries. The one or more predefined dictionaries may be provided in the database 43 (shown in FIG. 2) of the processor 30 of the medical coding system 12. In some embodiments, the one or more predefined dictionaries within the database 43 are semantic mapping tables. In some embodiments, the one or more predefined dictionaries use the United Medical Language System (UMLS) Metathesaurus, a biomedical thesaurus organized by concept.

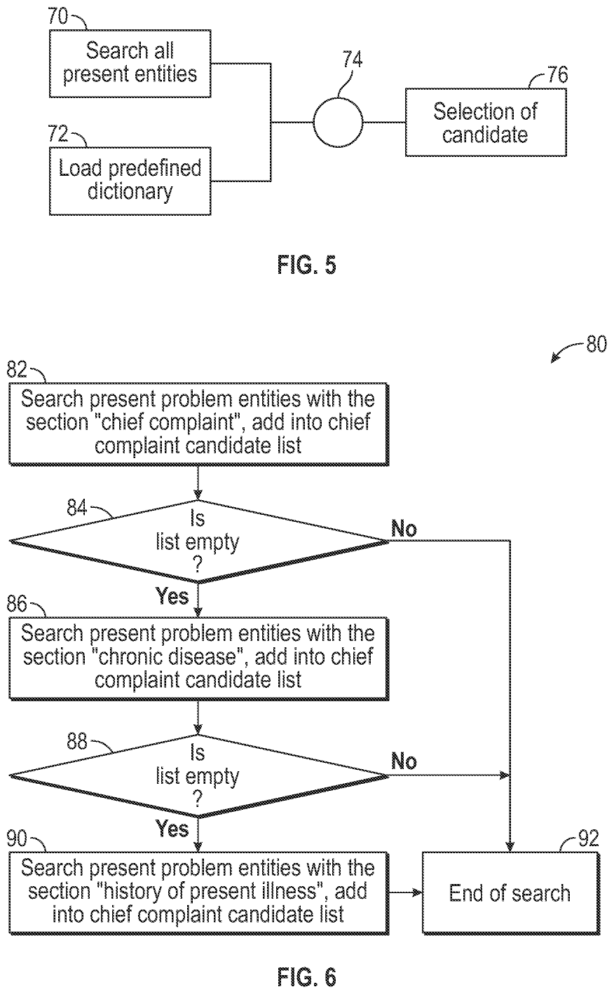

One or more predefined dictionaries may be developed and stored within the one or more database 43. Each predefined dictionary may include content needed for extraction from the clinical note 14 for determination of one or more candidates. For example, content in the clinical note 14 related to a chronic condition suffered by the patient 18 may be needed for determination of one or more candidates. To extract chronic conditions from the clinical note 14, a chronic conditions dictionary is developed and stored in the database 43 (e.g., non-transitory computer readable medium). The chronic conditions dictionary is developed by incorporating chronic conditions defined by Chronic Conditions Data Warehouse (CCW) and Unified Medical Language System (UMLS) concept unique identifiers (CUIs) retrieved from the UMLS Metathesaurus. The CUI is an identifier that uniquely represents a chronic condition, and generally, the CUI does not vary. For each CUI in the chronic condition dictionary, a pre-defined amount of concept CUIs are related to the chronic condition and stored. For example, in some embodiments, for each CUI, the chronic conditions dictionary relates and stores at least twenty-five concept CUIs. Referring to FIG. 5, the chronic conditions dictionary may be used to identify one or more chronic disease candidates. In a step 70, identified entities related to chronic conditions within the text of the clinical note 14 may be provided. In a step 72, the chronic disease dictionary may be provided and/or accessed. In a step 74, the identified entities related to chronic conditions within the clinical note 14 may be compared against the chronic disease dictionary. If the initial CUI of the chronic condition, or at least one of the concept CUIs for the chronic conditions is determined to be in the clinical note 14, the chronic condition is considered to be present in the clinical note 14 and considered a chronic disease candidate as shown in step 76 as shown in FIG. 3B.

In some embodiments, one or more predefined dictionaries stored in the database 43 may be a review of systems (ROS) dictionary. In some embodiments, the ROS dictionary may be configured to include terms and/or phrases of common disorders for each body system using UMLS concept CUIs. For each body system, common disorders may be determined. Further, for each common disorder determined, the ROS dictionary relates and stores a pre-determined amount of concept disorders. In some embodiments, for each common disorder, the ROS dictionary relates and stores at least twenty-five concept disorders to the initial common disorder. If the initial common disorder, or at least one of the concept disorders is determined to be in the clinical note 14, the common disorder is considered to be present in the clinical note 14. Referring to FIG. 5, the ROS dictionary, similar to the chronic conditions dictionary, may be used to identify one or more ROS candidates. In a step 70, identified entities related to ROS entities within the text of the clinical note 14 may be provided. In a step 72, the ROS dictionary may be provided and/or accessed. In a step 74, the identified entities related to ROS disorders within the clinical note 14 may be compared against the ROS dictionary. If the common disorder, or at least one of the concept disorders is determined to be in the clinical note 14, the common disorder is considered to be present in the clinical note 14 and considered a common disorder candidate as shown in step 76.

In some embodiments, one or more predefined dictionaries stored in the database 43 may be a human body dictionary. The human body dictionary may be configured to include terms and/or phrases for keywords within each body system. Keywords may be selected based on domain expertise (i.e., knowledge and/or understanding of essential aspects of each specific body system). The human body dictionary may also be used to classify which human body system is examined even when the healthcare provider 16 only documents diseases or problems within the clinical note 14. For example, if the healthcare provider 16 states “no fever, no fatigue,” the human body dictionary relates that the patient's constitutional system is sound, although “constitutional” is not textually specified within the clinical notes 14. In some embodiments, one or more predefined dictionaries stored in the database 43 may be a sentiment analysis keyword dictionary. The sentiment analysis keywords dictionary may be configured to perform via a sentiment analysis software, such as Valence Aware Dictionary for Sentiment Reasoning (VADER) Sentiment. Alterations may be made with the sentiment analysis keyword dictionary to conform to medical practice. For example, the terminology of the term “negative” within the medical field generally means no disorder exists; however, within current sentiment analysis software, the term “negative” is often assigned as a negative polarity value resulting in the failure to identify the medical connotation of the term “negative.” In another example, the sentiment analysis tool may be used as healthcare providers use adjective words to describe soundness of a patient body system, such as, “chest is clear.”

FIG. 6 illustrates a flow chart 80 of an exemplary method to provide a comprehensive candidate list in accordance with the present disclosure. The comprehensive candidate list provides review over multiple sections 40 of the clinical note 14. For example, the flow chart 80 of FIG. 6 is directed towards extracting chief complaint from the clinical note 14 shown in FIG. 3. Generally, extraction of the chief complaint may include review of section data 44 within multiple sections 40 within the clinical note 14. For example, extraction of the chief complaint may include review of the section data 44 within the chief complaint section, history of present illness section, chronic disease section, review of systems section, and the like. In some embodiments, during pre-classification, one or more entities may be recognized and extracted for review. In a step 82, one or more entities within the chief complaint section 40a may be searched and one or more chief complaint candidates may be identified. If one or more entities within the chief complaint section 40a is identified, the one or more entities may be added to the chief complaint candidate list and the search may end as shown in step 84. If the chief complaint candidate list is empty, one or more entities within the section 40 of “chronic disease” may be searched and one or more chief complaint candidates may be identified, as shown in step 86. If one or more entities within the section 40 of “chronic disease” is identified, the one or more entities may be added to the chief complaint candidate list and the search may end as shown in step 88. If the chief complaint candidate list is empty, one or more entities within the section 40 of “history of present illness” may be searched and one or more chief complaint candidates may be identified, as shown in step 90. If one or more entities within the section 40 of “history of present illness” is identified, the one or more entities may be added to the chief complaint candidate list and the search may end as shown in step 92. The method may continue for each section 40 within the clinical note 14 as needed.

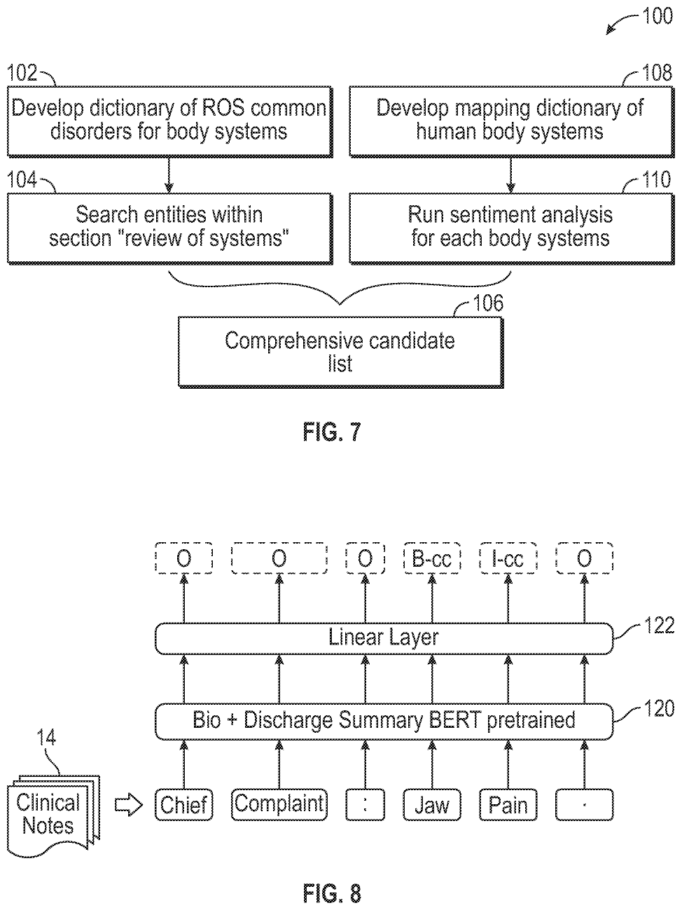

In some embodiments, multiple predefined dictionaries may be used to develop one or more candidates for the medical codes 20. FIG. 7 illustrates a flow chart 100 of an exemplary method of using multiple predefined dictionaries to determine one or more candidates for the medical codes 20. In a step 102, the ROS dictionary may be developed. In a step 104, one or more entities within the section of “review of systems” may be searched. In a step 106, identified entities within the section of “review of systems” may be added to a comprehensive candidate list. In a step 108, the mapping dictionary of human body system may be developed. In a step 110, sentiment analysis for each body system may be provided to identify entities and added to the comprehensive candidate list shown in step 106.

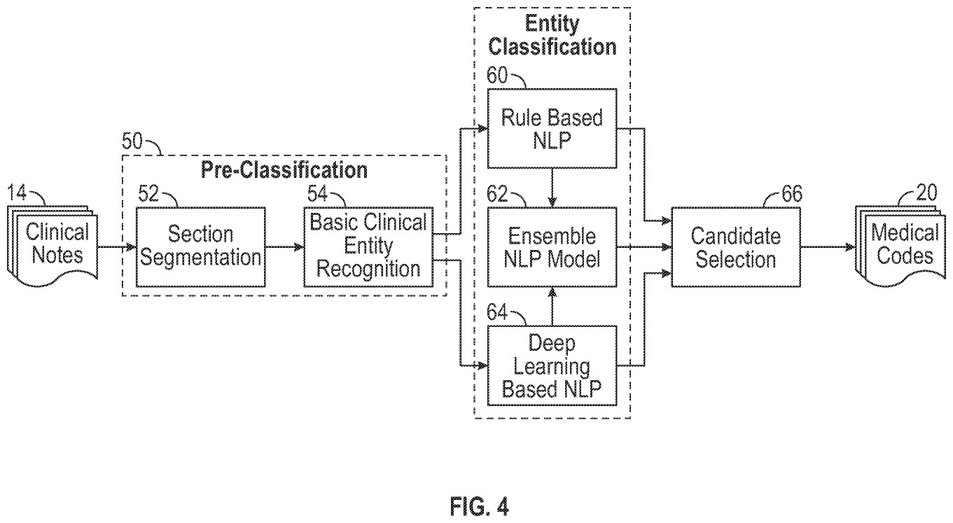

Referring to FIGS. 4 and 8, in some embodiments, a deep learning based NLP model 64 may be used to further aid in identification of one or more entities and development of one or more candidate lists to provide one or more medical codes 20 in accordance with the present disclosure. Generally, the deep learning based NLP model 64 is a transformer-based machine learning model for natural language processing known as Bidirectional Encoder Representations from Transformers (BERT). In some embodiments, the BERT model may be pre-trained using Medical Information Mart for Intensive Care-III (MIMIC-III) discharge summary notes configured to capture the context found in clinical notes 14 to provide a clinical BERT model 120. The clinical BERT model 120 may also be fine-tuned using a linear layer 122 to classify output of BERT into appropriate elements (e.g., patient history elements). The linear layer 122 may include a weighted matrix:

W∈ [symbol] ^{k×H} (EQ. 1)

wherein k is the total number of entity types. Representation as a vector is shown as:

C∈ [symbol] ^{H} (EQ. 2)

Classification may be solved by:

y=argmax(CW^{T}) (EQ. 3)

Results of the deep learning based NLP model 64 may be provided to the ensemble NLP model 62.

Referring to FIG. 4, the ensemble NLP model 62 may evaluate and aggregate the rule based NLP 60 and the deep learning based NLP model 64 resulting in increased accuracy of candidates 66. The ensemble NLP model 62 creates a new feature vector for each entity that is classified. For example, if the location of an entity is i, the vector is represented as:

$$( EQ . 4 )$$

$$v = concatnat {(}_{{prediction}_{i - 1}^{Deep - learning} , {prediction}_{i}^{Deep - learning} , {prediction}_{i + 1}^{Deep - learning}}^{{prediction}_{i - 1}^{Rule - based} , {prediction}_{i}^{Rule - based} , {prediction}_{i + 1}^{Rule - based} ,} )$$

wherein each prediction is a one-hot encoding of the candidate from the rule based NLP 60 and the corresponding prediction from the deep-learning based NLP model 64. The concatenate function is configured to combine the results into a single binary sequence. Then, vector V is entered into a support vector machine to build an ensemble classification model configured to recognize candidates 66 within the clinical notes 14 (i.e. the patient history elements including chief complaint, location, quality, severity, duration, timing, context, modifying factors, associated signs/symptoms, past history, family history, social history, and review of systems for fourteen body systems).

FIG. 3B illustrates exemplary output obtained from the exemplary clinical note 14 illustrated in FIG. 3A1-3 using the techniques described herein including natural language processing, expert knowledge of E/M coding protocols, rule-based algorithms, deep learning models, and/or an ensemble scheme as described in further detail herein. The output generally illustrates four elements of a patient history component used in E/M billing. Regarding the history list, the output includes binary numbers having values of 0 or 1. The numbers represent whether past medical history, family history and social history information exists (i.e., represented by value of 1), or does not exist (i.e., represented by value of 0). The review of systems (ROS) list includes fourteen different numbers, each with a value of 0, −1, or 1. Each of the fourteen numbers represents the health status of the fourteen review of systems defined in the 1997 Evaluation and Management Guidelines. For example, if information about a particular body system does not exist in the note, the number value is 0 for the health status of the particular body system. If there are no concerns or issues related to the particular body system within the clinical note 14, the number value is −1 for the health status of the particular body system. If concerns or issues exist within the clinical note 14 about the body system, the number value is 1 for the health status of the particular body system.

Referring to FIGS. 2 and 4, candidates 66 may be used to determine medical codes 20 using predefined rules stored within the database 43. Generally, the pre-defined rules include detailed official guidelines used within the medical billing industry such as the International Classification of Diseases (ICD) and the Current Procedural Terminology (CPT). CPT codes are five-digit alphanumeric codes used to identify services provided to patients such as medical, surgical, diagnostic, and radiological services. These codes are submitted with ICD-10 codes on claim forms to payers and used to determine reimbursement to a provider/facility. For example, according to Centers for Medicare and Medicaid Services (CMS) guidelines, a doctor visit with “Comprehensive” history component, “Comprehensive” examination component, and “High Risk” medical decision making component may be coded as CPT 99205. The pre-defined rules within the database 43 may identify each level of service (e.g., comprehensive, high risk). The guidelines for CPT 99205 states that a “Comprehensive” history component requires levels of History of Present Illness (HPI) to be “Extended”; Review of Systems (ROS) to be “Complete”; and, Past, Family and/or Social History (PFSH) to be “Complete.” Such guidelines are provided within the predefined rules stored within the database. In some embodiments, guidelines within the predefined rules stored within the database are provided in a hierarchical level. Candidates, as described herein, may be evaluated and based on the pre-defined rules (e.g., CMS guidelines), one or more medical codes 20 may be determined.

The following is a number list of non-limiting illustrative embodiments of the inventive concept disclosed herein:

1. A non-transitory computer readable medium storing computer readable instructions that when executed by a processor cause the processor to:
segment computer readable clinical notes into a plurality of sections having textual content; and
for each section:
parse the textual content with natural language processing using at least one pre-defined dictionary including at least one semantic mapping table; and, apply a set of predefined rules to the parsed textual content to generate at least one candidate
to provide at least one current procedural terminology (CPT) medical code based on the parsed textual content.

2. The non-transitory computer readable medium of claim 1, wherein at least one natural language processing (NLP) method is used to segment the computer readable clinical notes into a plurality of sections having textual content.

3. The non-transitory computer readable medium of claim 2, wherein the computer readable instructions identify at least one entity within the textual content of the section, the at least one entity comprised of at least one word referring to a term in a predefined category.

4. The non-transitory computer readable medium of claim 3, wherein at least one natural language processing (NLP) method is used to identify the least one entity in the textual content of the section.

5. The non-transitory computer readable medium of any one of claims 1-4, wherein the at least one pre-defined dictionary is configured using United Medical Language System (UMLS) Metathesaurus.

6. The non-transitory computer readable medium of claim 5, wherein the at least one pre-defined dictionary is a chronic conditions dictionary configured using chronic conditions defined by Chronic Conditions Data Warehouse (CCW) and UMLS concept unique identifiers (CUIs) retrieved from the UMLS Metathesaurus.

7. The non-transitory computer readable medium of claim 6, wherein the chronic conditions dictionary is configured by:
assigning a CUI as an identifier to uniquely represent a chronic condition; and,
assigning a pre-defined amount of concept CUIs related to the chronic condition to represent the chronic condition.

8. The non-transitory computer readable medium of claim 7, wherein identification of at least one concept CUI within the textual content indicates presence of the chronic condition within the textual content.

9. The non-transitory computer readable medium of claim 5, wherein the at least one pre-defined dictionary is a review of systems (ROS) dictionary configured to include terms of common disorders for each body system using UMLS concept unique identifiers (CUIs).

10. The non-transitory computer readable medium of claim 9, wherein the ROS dictionary is configured by:
assigning a CUI as an identifier to uniquely represent a common disorder; and,
assigning a pre-defined amount of concept CUIs to the common disorder to represent the common disorder.

11. The non-transitory computer readable medium of claim 10, wherein identification of at least one concept CUI within the textual content indicates presence of the common disorder within the textual content.

12. The non-transitory computer readable medium of claim 5, wherein at least one pre-defined dictionary is a human body dictionary configured to include keywords selected based on domain expertise.

13. The non-transitory computer readable medium of claim 12, wherein at least one pre-defined dictionary is a sentiment analysis keyword dictionary conforming to medical practice.

14. The non-transitory computer readable medium of any one of claims claim 1-13, wherein the computer executable instructions define a hierarchical process to extract chief complaint candidates from different sections.

15. The non-transitory computer readable medium of any one of claims 1-13, wherein the computer readable instructions perform a deep learning model to identify at least one candidate for determination of the CPT medical code.

16. The non-transitory computer readable medium of claim 15, wherein the deep learning model is based on bidirectional encoder representation from transformers (BERT) pre-trained on Medical Information Mart for Intensive Care (MIMIC)-III discharge summary notes.

17. The non-transitory computer readable medium of claim 16, wherein the computer readable instructions perform an ensemble scheme configured to aggregate results of the set of predefined rules and the deep learning model to provide at least one candidate.

18. A non-transitory computer readable medium storing computer readable instructions that when executed by a processor cause the processor to:
preclassify a computer readable clinical note by:
segmenting the computer readable clinical notes into a plurality of sections having textual content; and
identifying a plurality of entities within each section, each entity being at least one word referring to a term in a predefined category;
for each section:
parse the textual content with natural language processing using at least one pre-defined dictionary including at least one semantic mapping table; and,
apply a set of predefined rules to the parsed textual content to generate at least one predefined rules candidate;
perform a deep learning model to identify at least one deep learning model candidate;
perform an ensemble scheme configured to aggregate results of the set of predefined rules and the deep learning model to provide at least one ensemble candidate; and,
provide at least one current procedural terminology (CPT) medical code based on the at least one ensemble candidate.

19. The non-transitory computer readable medium of claim 18, wherein the at least one pre-defined dictionary is a chronic conditions dictionary configured using chronic conditions defined by Chronic Conditions Data Warehouse (CCW) and United Medical Language System (UMLS) concept unique identifiers (CUIs) retrieved from a UMLS Metathesaurus, the chronic conditions dictionary configured by:
assigning a CUI as an identifier to uniquely represent a chronic condition; and,
assigning a pre-defined amount of concept CUIs related to the chronic condition to represent the chronic condition.

20. A method, comprising:
segmenting a computer readable clinical note into a plurality of sections having textual content;
identifying a plurality of entities within each section, each entity being at least one word referring to a term in a predefined category;
parsing textual content for each section with natural language processing using at least one pre-defined dictionary including at least one semantic mapping table;
applying a set of predefined rules to the parsed textual content to generate at least one predefined rules candidate;
performing a deep learning model on the computer readable computer note to identify at least one deep learning model candidate;
performing an ensemble scheme configured to aggregate results of the set of predefined rules and the deep learning model to provide at least one ensemble candidate; and,
provide at least one current procedural terminology (CPT) medical code based on the at least one ensemble candidate.

From the above description, it is clear that the inventive concepts disclosed and claimed herein are well adapted to carry out the objects and to attain the advantages mentioned herein, as well as those inherent in the invention. While exemplary embodiments of the inventive concepts have been described for purposes of this disclosure, it will be understood that numerous changes may be made which will readily suggest themselves to those skilled in the art and which are accomplished within the spirit of the inventive concepts disclosed and claimed herein.

What is claimed is:

1. A non-transitory computer readable medium storing computer readable instructions that when executed by a processor cause the processor to:
segment computer readable clinical notes into a plurality of sections having textual content; and
for each section:
parse the textual content with natural language processing using at least one pre-defined dictionary including at least one semantic mapping table; and,
apply a set of predefined rules to the parsed textual content to generate at least one candidate to provide at least one current procedural terminology (CPT) medical code based on the parsed textual content.

2. The non-transitory computer readable medium of claim 1, wherein at least one natural language processing (NLP) method is used to segment the computer readable clinical notes into a plurality of sections having textual content.

3. The non-transitory computer readable medium of claim 2, wherein the computer readable instructions identify at least one entity within the textual content of the section, the at least one entity comprised of at least one word referring to a term in a predefined category.

4. The non-transitory computer readable medium of claim 3, wherein at least one natural language processing (NLP) method is used to identify the least one entity in the textual content of the section.

5. The non-transitory computer readable medium of claim 1, wherein the at least one pre-defined dictionary is configured using United Medical Language System (UMLS) Metathesaurus.

6. The non-transitory computer readable medium of claim 5, wherein the at least one pre-defined dictionary is a chronic conditions dictionary configured using chronic conditions defined by Chronic Conditions Data Warehouse (CCW) and UMLS concept unique identifiers (CUIs) retrieved from the UMLS Metathesaurus.

7. The non-transitory computer readable medium of claim 6, wherein the chronic conditions dictionary is configured by:
assigning a CUI as an identifier to uniquely represent a chronic condition; and,
assigning a pre-defined amount of concept CUIs related to the chronic condition to represent the chronic condition.

8. The non-transitory computer readable medium of claim 7, wherein identification of at least one concept CUI within the textual content indicates presence of the chronic condition within the textual content.

9. The non-transitory computer readable medium of claim 5, wherein the at least one pre-defined dictionary is a review of systems (ROS) dictionary configured to include terms of common disorders for each body system using UMLS concept unique identifiers (CUIs).

10. The non-transitory computer readable medium of claim 9, wherein the ROS dictionary is configured by:
assigning a CUI as an identifier to uniquely represent a common disorder; and,
assigning a pre-defined amount of concept CUIs to the common disorder to represent the common disorder.

11. The non-transitory computer readable medium of claim 10, wherein identification of at least one concept CUI within the textual content indicates presence of the common disorder within the textual content.

12. The non-transitory computer readable medium of claim 5, wherein at least one pre-defined dictionary is a human body dictionary configured to include keywords selected based on domain expertise.

13. The non-transitory computer readable medium of claim 12, wherein at least one pre-defined dictionary is a sentiment analysis keyword dictionary conforming to medical practice.

14. The non-transitory computer readable medium of claim 1, wherein the computer executable instructions define a hierarchical process to extract chief complaint candidates from different sections.

15. The non-transitory computer readable medium of claim 1, wherein the computer readable instructions perform a deep learning model to identify at least one candidate for determination of the CPT medical code.

16. The non-transitory computer readable medium of claim 15, wherein the deep learning model is based on bidirectional encoder representation from transformers (BERT) pre-trained on Medical Information Mart for Intensive Care (MIMIC)-III discharge summary notes.

17. The non-transitory computer readable medium of claim 16, wherein the computer readable instructions perform an ensemble scheme configured to aggregate results of the set of predefined rules and the deep learning model to provide at least one candidate.

18. A non-transitory computer readable medium storing computer readable instructions that when executed by a processor cause the processor to:
preclassify a computer readable clinical note by:
segmenting the computer readable clinical notes into a plurality of sections having textual content; and
identifying a plurality of entities within each section, each entity being at least one word referring to a term in a predefined category;
for each section:
parse the textual content with natural language processing using at least one pre-defined dictionary including at least one semantic mapping table; and,
apply a set of predefined rules to the parsed textual content to generate at least one predefined rules candidate;
perform a deep learning model to identify at least one deep learning model candidate;
perform an ensemble scheme configured to aggregate results of the set of predefined rules and the deep learning model to provide at least one ensemble candidate; and,
provide at least one current procedural terminology (CPT) medical code based on the at least one ensemble candidate.

19. The non-transitory computer readable medium of claim 18, wherein the at least one pre-defined dictionary is a chronic conditions dictionary configured using chronic conditions defined by Chronic Conditions Data Warehouse (CCW) and United Medical Language System (UMLS) concept unique identifiers (CUIs) retrieved from a UMLS Metathesaurus, the chronic conditions dictionary configured by:
assigning a CUI as an identifier to uniquely represent a chronic condition; and,
assigning a pre-defined amount of concept CUIs related to the chronic condition to represent the chronic condition.

20. A method, comprising:
segmenting a computer readable clinical note into a plurality of sections having textual content;
identifying a plurality of entities within each section, each entity being at least one word referring to a term in a predefined category;
parsing textual content for each section with natural language processing using at least one pre-defined dictionary including at least one semantic mapping table;
applying a set of predefined rules to the parsed textual content to generate at least one predefined rules candidate;
performing a deep learning model on the computer readable computer note to identify at least one deep learning model candidate;
performing an ensemble scheme configured to aggregate results of the set of predefined rules and the deep learning model to provide at least one ensemble candidate; and,
provide at least one current procedural terminology (CPT) medical code based on the at least one ensemble candidate.
